(19) United States
(12) Patent Application Publication
(10) Pub. No.: US 2007/0194100 A1
(43) Pub. Date: Aug. 23, 2007

(54) REMOTE CONTROL SYSTEM FOR SEQUENCE CONTROL IN MEDICAL TREATMENT...

(75) Inventors: Norman Plassky, Erfurt (DE); Timo Neubauer, Poing/Angelbrechting (DE)

Correspondence Address: DON W. BULSON (BrainLAB), RENNER, OTTO, BOISSELLE & SKLAR, LLP, 1621 EUCLID AVENUE - 19TH FLOOR, CLEVELAND, OH, 44115, US

(73) Assignee: BRAINLAB AG, Feldkirchen (DE)

(21) Appl. No.: 11/674,383

(22) Filed: Feb. 13, 2007

Related U.S. Application Data
(60) Provisional application No. 60/777,074, filed on Feb. 27, 2006.

(30) Foreign Application Priority Data
Feb. 14, 2006 (EP) 06002904

(51) Int. Cl. G06F 17/00; G05B 19/00; G06F 7/00; G08B 13/14

(52) U.S. Cl. 235/375; 700/225; 340/572.1; 340/5.1

(57) ABSTRACT

A remote control system for sequence control in medical treatment-assisting systems includes a signal emitter including at least one switchable transponder operative to generate a signal, said signal emitter remotely located relative to the treatment-assisting system, and a signal receiver operable to process the emitted signal within the framework of sequence control. The signal receiver is assigned to a transmitter/receiver unit that operates using radio-frequency identification (RFID) technology.

### RELATED APPLICATION DATA

This application claims priority of U.S. Provisional Application No. 60/777,074 filed on Feb. 27, 2006, which is incorporated herein by reference in its entirety.

### FIELD OF THE INVENTION

The invention relates to a remote control system and method for sequence control in medical treatment-assisting systems. More particularly, the invention relates to a remote control system and method that include a signal emitter arranged a distance from a treatment-assisting system, and a signal receiver that processes signals from the signal emitter within the framework of sequence control.

### BACKGROUND OF THE INVENTION

When using treatment-assisting systems in the field of medical technology, in particular when using image-assisting systems or navigation systems, decisions regarding a subsequent course or courses of treatment assistance are made by a member of the operating team. For example, a step may have to be repeated or, depending on the circumstances, there may be several options for the subsequent course of treatment, wherein one option must be selected. For these purposes, a signal emitter often is used that relays a signal to a signal receiver. The signal receiver is assigned to the treatment-assisting system so as to make or otherwise select a course of action within the sequence.

Various and different solutions have been proffered for generating and transmitting the signal, e.g., providing foot switches connected to the treatment-assisting system via cables. Wireless transmission also may be provided by means of a pointer (surgical probe), for example, which can be used interactively to point to a navigation screen. Since a camera of the navigation system is rigidly attached to the screen, the commands, which may be displayed on the screen as switches, can be selected using a virtually extended pointer tip.

Moving markers also may be used on treatment apparatus (pointers), which when moved or after having been moved trigger a data input, e.g., the initiation of a registration process. Other solutions include pressing a button on an instrument in order to switch on a specific LED or LED array, which is visible to the navigation system. The navigation system identifies the illumination as a data input.

US 2005/0228266 A1 describes a system comprising a marker array that includes predetermined locations or zones which, when a probe tip is moved onto the predetermined locations or zones, a command is relayed to the navigation system.

WO 98/08062 discloses the use of activating means that can be attached to the fingers of a person carrying out the treatment. The activating means generate signals that can in turn operate a sequence controller (e.g., computer program execute by a processor).

A drawback to such prior art systems is that they are complicated to operate or evaluate, or are a strain on the user's attention.

### SUMMARY OF THE INVENTION

In accordance with the present invention, a remote control system and method utilizes a signal emitter having at least one switchable transponder, and a signal receiver assigned to a transmitter/receiver unit that operates using radio-frequency identification (RFID) technology. In other words, a localization and identification system for signal emitters can be used that is not dependent on either a cable connection to the treatment-assisting system or visibility of specific navigation marker arrays, i.e., an existing line of sight.

Since switchable transponders are simple to operate, such “secondary operations” also are not an undue strain on the attention of the person carrying out the treatment. Within the framework of the invention, the treatment-assisting system and method can be a medical image-assisting system, in particular a medical navigation system.

The signal emitter can include several transponders that are arranged on a transponder support and are suitable for emitting different signals (e.g., different commands). An embodiment with only one switchable transponder, however, is of course also conceivable.

There exists the option of configuring the transponder support such that it is portable and easily managed, e.g., a roughly hand-sized mat on which the transponder/transponders is/are arranged for operation. The transponder support and the transponder/transponders can be configured as a disposable item; however, it is of course also possible to employ a reusable transponder support which is formed as a unit that can be sterilized or re-sterilized.

The transponder support can include a bonding device, in particular a non-permanent adhesive layer or a material bonding device, e.g., a hook and loop material such as a Velcro® device. If this bonding device is attached on the reverse side of the transponder support, i.e., on the side on which no switchable transponders are arranged, the support can be easily secured at various locations, which improves its operability.

Although such transponders in RFID systems do not usually require an energy supply, an internal energy generating device can be provided for the transponder support in order to ensure a sufficient energy supply to operate the transponder. The energy supply can comprise, for example, batteries or power packs, a solar energy supply, or the like.

The transponder support can be a treatment instrument or treatment-assisting instrument, in particular a surgical probe (pointer). The transmitter/receiver unit can be assigned to the treatment-assisting system, in particular arranged on or in one of the parts of the system.

In a method for sequence control in medical treatment-assisting systems, a signal receiver processes a signal provided by a signal emitter distanced from the system, within the framework of sequence control, wherein the signal is transmitted by means of a signal emitter that includes at least one switchable transponder, and wherein the signal receiver is assigned to a transmitter/receiver unit that operates using radio-frequency identification (RFID) technology. The features described herein can of course all be implemented in a method for sequence control. The transmitter/receiver unit may be switched on only when the sequence control and/or the assisted course of the treatment provides for processing the signal from the signal emitter.

In other words, a remote control means may be provided that allows a computer program to be operated using wireless transmission technology. The remote control can not only be attached to the patient or to the person carrying out the treatment, but on any other surface in the treatment area, for example on a table or other instruments. The remote control is advantageously a passive device and not dependent on energy being supplied by batteries, as the radiation energy from the transmitter/receiver unit can be used for operation, and the transponders only provide a resonance to the generated alternating field once the transponder has been switched on. If the remote control comprises only one transponder and/or transponder push button, the transponder and/or transponder push button can transmit a signal that provides notification that an instrument has been activated or deactivated. Different transponder push buttons having different predefined commands, however, can be used to precisely control the software sequence.

The transponder can include a circuit that causes the transponder to switch on when operated and to switch off again once released. The internal information of the transponders, which may be different for each function, can be read by the transmitter/receiver unit and then relayed to the treatment-assisting system (e.g., a navigation system), where the information is interpreted as a command. In order to enable command inputs to be captured, the transmitter/receiver unit preferably is activated by the navigation and/or assisting software whenever (and preferably only when) a command input is indeed possible or allowed. This can reduce the emission of radiation in the area. The transmitter/receiver unit can in principle be arranged such that it allows transponder localization anywhere in the treatment area, but is preferably assigned to the treatment-assisting system where it is provided on or in the casing and/or on or in the assigned camera system.

One advantage relative to systems that operate using virtually extended pointer tips and switches on the screen is that it is possible to have the remote control permanently in the treatment area, without having to use the pointer (the pointer can be used for other purposes or, once the appropriate method steps have been completed, can be removed from the operating area). This increases the clarity of inspection in the working area. It is also more versatile in its use, since the transponder push buttons are easy to locate and easy to operate. Compared to the cable-connected signal transmission systems (e.g., foot switches), there exists the advantage that several different switches, in particular more than two or three, can be provided that are clearly designated and therefore easy to locate, without having to search for the switch in each case. The absence of cables naturally helps to keep the treatment area clear for inspection, clean and to avoid accidents. If it is suitably positioned, the remote control can be operated by treatment staff standing on any side of the patient.

The advantages, already mentioned above, which come from the unrestricted use of the corresponding treatment apparatus also come to bear with respect to the markers and/or marker arrays from the prior art that can be moved or activated. One advantage in this respect is that it is immaterial whether the transponders and/or transponder arrays are visible to the tracking and/or navigation system, thereby allowing the remote control to be used more flexibly within the treatment area. Also, with marker arrays that can be activated or altered, the functionality is significantly limited to a small number of signals and/or commands while individual markers or all markers are altered or exchanged, whereas the transmission technology enables a greater number of different transmitted signals, without requiring highly complex mechanical devices.

The device can be manufactured as a cost-effective reusable product, or even as a disposable product, and enables the treatment staff to influence the control sequence without using costly or complicated equipment, such as touch-sensitive screens, keyboards/mouse systems.

### BRIEF DESCRIPTION OF THE DRAWINGS

The forgoing and other features of the invention are hereinafter discussed with reference to the drawings.

FIG. 1 illustrates an exemplary operating theater set-up with separate navigation and tracking systems, wherein an exemplary remote control system in accordance with the invention is used.

FIG. 2 illustrates an exemplary operating theater set-up with an integrated tracking/navigation system and an exemplary remote control system in accordance with the invention.

FIG. 3 is a partial section view (top) of an exemplary transponder support in accordance with the invention.

FIGS. 4a and 4b illustrates an exemplary transponder push button in an isolated representation in accordance with the invention.

FIG. 5 illustrates an exemplary transponder switch on a surgical pointer in accordance with the invention.

### DETAILED DESCRIPTION

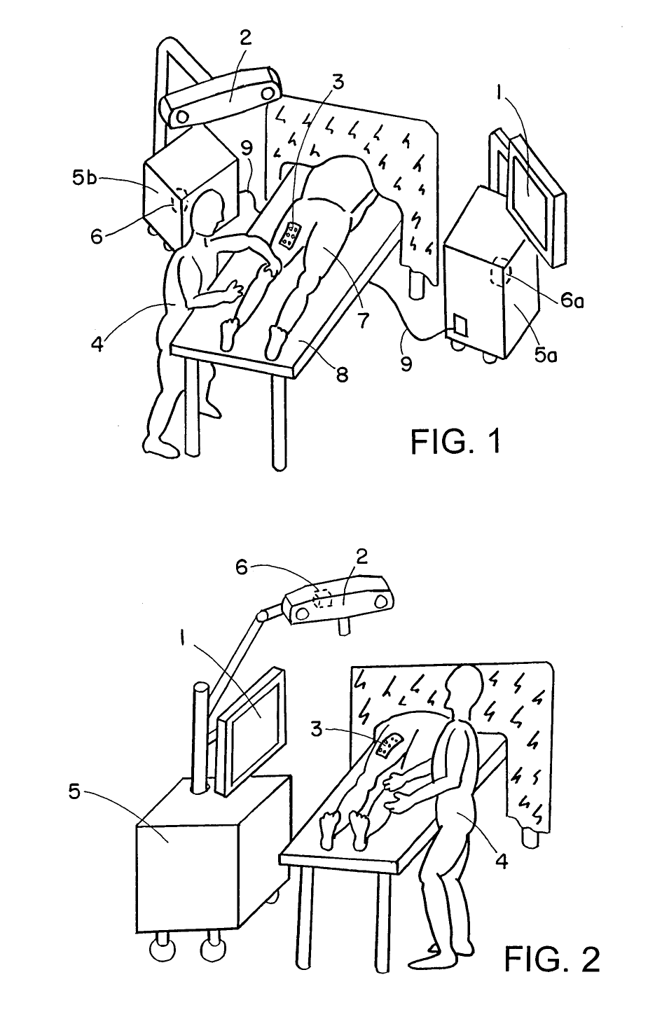

The devices shown in FIGS. 1 and 2 include: a navigation system screen 1, such as a touch screen or the like; a camera tracking unit 2; a transponder mount 3, also referred to below as a remote control unit, mat or pad; a surgeon 4; an integrated navigation system 5 (e.g., a combined navigation unit and a tracking unit), wherein 5a and 5b represent a separate arrangement of the navigation unit 5a and tracking unit 5b; an RFID transmitter/receiver unit 6; an RFID transmitter/receiver unit 6a which merely by way of example is shown arranged at a different location (on the separate navigation system); a patient 7; and lastly an operating table 8 on which the patient 7 lies.

As used herein, the terms “remotely mounted” or “remotely located” are defined as positioning a first device relative to a second device (including the device itself as well as any I/O stations of the second device) such that when an average individual is located at the first device, he cannot, without assistance, reach out and touch the second device.

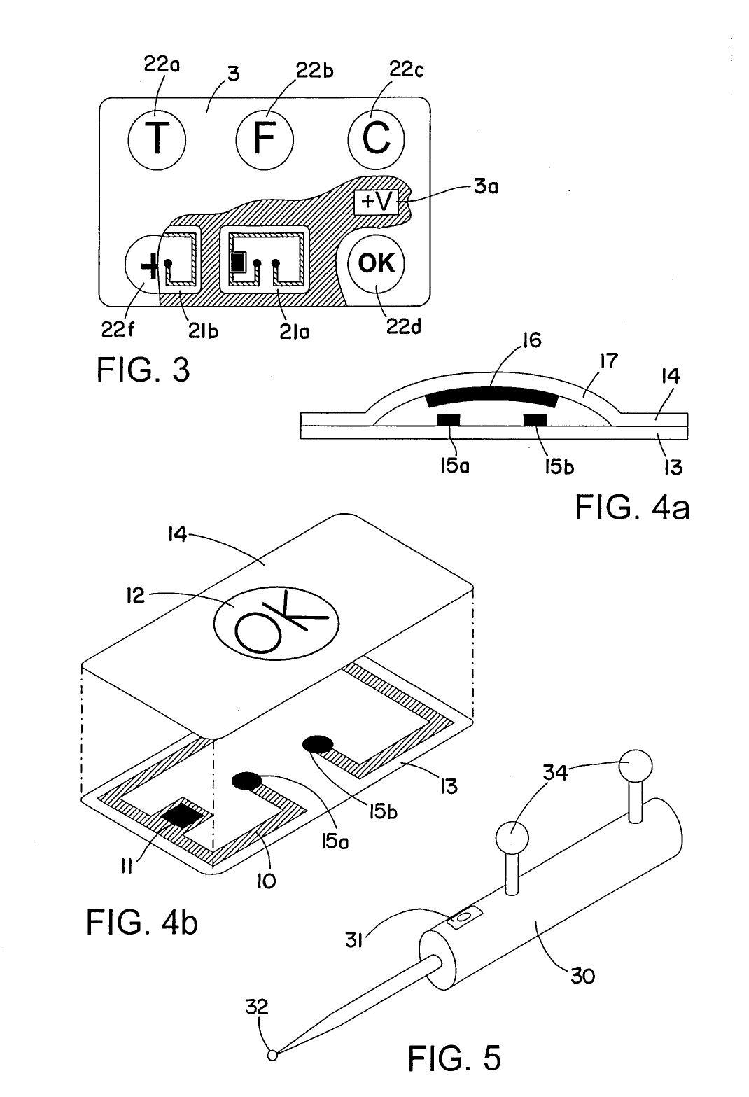

Using the device and method described herein, it is possible to operate software of the navigation system 5, 5a, 5b from a location at which the surgeon 4 is not capable of making a direct input into the navigation system (for example, using the touch screen 1). To this end, the device can include a wireless remote control unit 3 (also referred to as a transponder mount), which may be configured as a small mat or pad and comprises a transponder support having switchable transponders. The mat 3 can be made of a flexible plastic, and is described in more detail below with respect to FIGS. 3 and 4. The transmitter/receiver unit 6 shown in FIG. 1 can be arranged on the tracking unit 5b (e.g., on the camera holder) of the navigation system; an alternative embodiment is however also shown, comprising a transmitter/receiver unit 6a on the navigation unit 5a. If a system separated in this way is provided, it can transmit signals and/or communicate using a cable connection 9, as shown in FIG. 1.

Referring to FIG. 2, the transmitter/receiver unit 6 is provided at a different location from that of FIG. 1, namely in the casing of the tracking camera unit 2. It is noted, however, that the transmitter/receiver unit 6 can be positioned at any suitable location within the treatment area.

The remote control unit 3 can be an independent (i.e., no connecting cable) and passive device that can operate without a battery or power pack supply, as it can draw energy from the alternating field of the transmitter/receiver unit 6. A power supply 3a, such as a solar supply or other power supply, however, can be provided on the remote control itself, as a backup or for particularly energy-intensive switchable transponders. The remote control unit 3 can be attached to any surface in the treatment area, or for example to the sleeve of the surgeon 4 or another member of the operating team. In FIG. 1, the remote control unit 3 is shown attached to the leg of the patient 7, which proves advantageous since other members of the operating team also then have access to the switching functions. For this purpose, the remote control unit 3 can have a bonding reverse side (for example, a non-permanent adhesive layer or Velcro® fastening).

Referring to FIGS. 3 and 4a-4b, the energy emitted by the transmitter/receiver unit 6 in the form of electromagnetic field radiation can be used for data processing within a transponder microchip 11 and for transmitting a response signal to the transmitter/receiver unit 6. The transponders and their arrangement within the flexible plastic pad which serves as the remote control unit (transponder support) 3 can be seen from FIGS. 3 and 4a-4b. In order to transmit various switching signals and therefore various commands, the remote control unit 3 can include a number of push buttons 22a to 22f connected to RFID resonance circuits 21a to 21f. As will be appreciated, any circuit can be used that is capable of operating as a transponder.

The transponders can be switchable transponders, which means that the circuits 10 of the transponders 21a to 21f are open at a particular location in their initial state, but can be closed by the electrodes 15a, 15b when the connecting button 12, which comprises a conductive layer 16 on its underside that acts as a circuit bridge, is pressed. In the area of the pad and/or mat, the switchable transponder includes a lower layer 13 and an upper layer 14. The upper layer can include at least one flexible region 17 that comprises the conductive layer 13 on its underside. By merely pressing the button 12, it is possible to transmit a response signal and/or switching signal to the transmitter/receiver unit 6, once the software of the navigation system has activated said transmitter/receiver unit 6, such that it can receive an input command. Closing the circuit 10 via the electrodes 15a and 15b and the bridge 16 thus completes the resonance circuit of the transponder.

All the other buttons that are not pressed are inactive or “quiet”, i.e., they do not transmit a signal, since they do not resonate. The information transmitted by the transponders may be different for each transponder button 22a to 22f, in order to allow the navigation system and/or navigation software to differentiate between different signals and, therefore, influence the sequence in the desired way. Using such a device, many different signals can be transmitted in a simple way.

Referring to FIG. 5, a device is shown wherein only a single switchable transponder 31 is fixed to a pointer 30, wherein the pointer 30 is localized in the navigation system via a marker array 34. This enables a position of the pointer tip 32 to be ascertained and displayed. For such an individual instrument, it is often sufficient to provide only a single push button and/or transponder button 31 that can be fixed to the instrument. By activating the transponder, it is possible to intervene in sequence control, for example by indicating to the navigation system (e.g., by pressing button 31) that said pointer has now been activated and is being used in the system and that additional operations are therefore possible. The small, separately fixable mat comprising the transponder switch 31 enables this without compromising the stability, weight, complexity or ease of handling of the instrument 30.

Although the invention has been shown and described with respect to a certain preferred embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

What is claimed is:

1. A remote control system for sequence control in medical treatment-assisting systems, comprising:
a signal emitter including at least one switchable transponder operative to generate a signal, said signal emitter remotely located relative to the treatment-assisting system; and
a signal receiver operable to process the emitted signal within the framework of sequence control, wherein the signal receiver is assigned to a transmitter/receiver unit that operates using radio-frequency identification (RFID) technology.

2. The remote control system according to claim 1, wherein the treatment-assisting system is a medical navigation system.

3. The remote control system according to claim 1, wherein the signal emitter includes a plurality of transponders arranged on a transponder mount, said transponders operable to emit different signals.

4. The remote control system according to claim 3, wherein the signal emitter includes a transponder mount comprising a portable mat on which the at least one switchable transponder is arranged.

5. The remote control system according to claim 4, wherein the transponder mount and the at least one transponder are configured as a disposable item.

6. The remote control system according to claims 4, wherein the transponder mount comprises a non-permanent bonding device.

7. The remote control system according to claim 6, wherein the non-permanent bonding device is a hook and loop bonding device.

8. The remote control system according to claim 4, wherein the transponder mount comprises an energy generating device.

9. The remote control system according to claim 8, wherein the energy generating device is a solar energy supply.

10. The remote control system according to claim 4, wherein transponder mount is a sterilizable unit.

11. The remote control system according to claim 1, wherein the transponder mount is a treatment instrument or treatment-assisting instrument

12. The remote control system according to claim 11, wherein the treatment instrument is a surgical probe or pointer.

13. The remote control system according to claim 1, wherein the transmitter/receiver unit is arranged on or in the treatment assisting system, or on or in parts of the treatment assisting system.

14. A method for sequence control in medical treatment-assisting systems, comprising:
using a signal emitter to generate a signal indicative of a desired command, said signal emitter including at least one switchable transponder to transmit the signal, wherein the signal emitter is remotely located relative to the medical treatment-assisting system; and
using a signal receiver to receive and process the signal generated by a signal emitter, wherein the signal receiver is assigned to a transmitter/receiver unit that operates using a radio-frequency identification (RFID) technology.

15. The method according to claim 14, further comprising switching on the transmitter/receiver unit only when the sequence control and/or the assisting course of the treatment provides for processing the signal from the signal emitter.
